SEQUENCE LISTING (3 sequences)

SEQ ID NO: 1 - protein, 301 aa, Artificial Sequence
  made in laboratory
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 2 - protein, 605 aa, Artificial Sequence
  made in laboratory
VISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINYGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 3 - protein, 605 aa, Artificial Sequence
  made in laboratory
VISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

(19) United States
(12) Patent Application Publication
(10) Pub. No.: US 2024/0218355 A1
(43) Pub. Date: Jul. 4, 2024

(54) DNA END REPAIR REAGENT AND KIT THEREOF, DNA LIBRARY CONSTRUCTION KIT, AND METHOD FOR CONSTRUCTING DNA LIBRARY

(71) Applicants: CHENGDU BOE OPTOELECTRONICS TECHNOLOGY CO., LTD., Sichuan (CN); BOE TECHNOLOGY GROUP CO., LTD., Beijing (CN)

(72) Inventor: Bangquan YE, Beijing (CN)

(73) Assignees: CHENGDU BOE OPTOELECTRONICS TECHNOLOGY CO., LTD., Sichuan (CN); BOE TECHNOLOGY GROUP CO., LTD., Beijing (CN)

(21) Appl. No.: 17/922,049

(22) PCT Filed: Nov. 9, 2021
(86) PCT No.: PCT/CN2021/129659

§ 371 Date: Oct. 28, 2022

(51) Int. Cl. C12N 15/10

(52) U.S. Cl. CPC C12N 15/1068; C12N 15/1072

(57) ABSTRACT

A deoxyribonucleic acid (DNA) end repair reagent includes: a DNA end repair combinatorial enzyme and a single strand DNA-binding protein (SSB).

### CROSS-REFERENCE TO RELATED APPLICATION

This application is a national phase entry under 35 USC 371 of International Patent Application No. PCT/CN2021/129659 filed on Nov. 9, 2021 which is incorporated herein by reference in its entirety.

### TECHNICAL FIELD

The present disclosure relates to the field of biotechnologies, and in particular, to a DNA end repair reagent and kit thereof, a DNA library construction kit and a method for constructing a DNA library.

### BACKGROUND

A deoxyribonucleic acid (DNA) library refers to a collection including all expressible gene segments in a genome of a certain organism. Considering a high-throughput sequencing (HTS) platform as an example, an existing routine process for constructing a library is: fragmenting genomic DNA, performing end repair and A (adenine) addition on formed DNA segments, ligating sequencing adapters to the DNA segments each with A, and finally enriching and purifying adapter ligation products to complete the construction of the library.

### SUMMARY

In one aspect, a DNA end repair reagent is provided. The DNA end repair reagent includes: a DNA end repair combinatorial enzyme and a single strand DNA-binding protein (SSB).

In some embodiments, the SSB is a T4 gene 32 protein.

In some embodiments, an amino acid sequence of the SSB is as shown in a sequence 1 in a sequence listing.

In some embodiments, a concentration of the SSB in the DNA end repair reagent is in a range of 0.5 μg/μL to 2 μg/μL, inclusive.

In some embodiments, the DNA end repair combinatorial enzyme includes: an enzyme I having a 5′-3′ DNA polymerase activity and a 3′-5′ DNA exonuclease activity.

In some embodiments, the enzyme I includes a Klenow fragment; or the enzyme I includes a mutant of the Klenow fragment.

In some embodiments, an amino acid sequence of the mutant of the Klenow fragment is as shown in a sequence 2 in the sequence listing.

In some embodiments, in a case where the enzyme I in the DNA end repair combinatorial enzyme includes the Klenow fragment, a concentration of the Klenow fragment in the DNA end repair reagent is in a range of 0.02 U/μL to 0.15 U/μL, inclusive; and in a case where the enzyme I in the DNA end repair combinatorial enzyme includes the mutant of the Klenow fragment, a concentration of the mutant of the Klenow fragment in the DNA end repair reagent is in a range of 0.02 U/μL to 0.15 U/μL, inclusive.

In some embodiments, the DNA end repair reagent further includes: a polyethylene glycol (PEG), the PEG being selected from one or more of a PEG-4000, a PEG-6000 and a PEG-8000.

In some embodiments, a mass percentage of the PEG in the DNA end repair reagent is in a range of 8% to 25%, inclusive.

In some embodiments, the DNA end repair reagent further includes a dNTP. The DNA end repair combinatorial enzyme further includes an enzyme II capable of adding A to a 3′ end of DNA and an enzyme III capable of phosphorylating a 5′ end of the DNA.

In some embodiments, enzyme II includes a Taq DNA polymerase, and the enzyme III includes a T4 polynucleotide kinase (T4 PNK).

In another aspect, a deoxyribonucleic acid (DNA) end repair kit is provided. The DNA end repair kit includes: the DNA end repair reagent described above.

In yet another aspect, a deoxyribonucleic acid (DNA) library construction kit is provided. The DNA library construction kit includes: the DNA end repair kit described above, and the DNA adapter ligation kit described above.

In some embodiments, a mass percentage of the PEG-4000 in the DNA adapter ligation reagent is in a range of 8% to 25%, inclusive.

In yet another aspect, a method for constructing a DNA library is provided. The method includes:
fragmenting genomic DNA to obtain first DNA fragments;
treating the first DNA fragments by using the DNA end repair reagent according to any one of claims 1 to 10 to obtain second DNA fragments, the second DNA fragments being each a fragment which is phosphorylated at 5′ ends and with adenine (A) at 3′ ends, wherein a treatment condition is: firstly treating at 15° C. to 25° C. for 10 min to 20 min, and then treating at 60° C. to 70° C. for 10 min to 20 min;
ligating sequencing adapters to the second DNA fragments to obtain adapter ligation products; and
purifying the adapter ligation products, and enriching the purified products.

In some embodiments, ligating the sequencing adapters to the second DNA fragments, includes:
treating the second DNA fragments by using the DNA adapter ligation reagent described above, so as to ligate the sequencing adapters to the second DNA fragments.

### BRIEF DESCRIPTION OF THE DRAWINGS

In order to describe technical solutions in the present disclosure more clearly, accompanying drawings to be used in some embodiments of the present disclosure will be introduced briefly below. However, the accompanying drawings to be described below are merely accompanying drawings of some embodiments of the present disclosure, and a person having ordinary skill in the art can obtain other drawings according to these accompanying drawings. In addition, the accompanying drawings in the following description may be regarded as schematic diagrams, but are not limitations on an actual size of a product, an actual process of a method and an actual timing of a signal involved in the embodiments of the present disclosure.

FIG. 1A is a flow chart of a 5′-3′ synthesis reaction of DNA, in accordance with some embodiments;

FIG. 1B is a flow chart of a 3′-5′ excision reaction of DNA, in accordance with some embodiments;

FIG. 2 is a flowchart of a method for constructing a DNA library, in accordance with some embodiments; and

FIG. 3 is a structural diagram of a Y-shaped adapter, in accordance with some embodiments.

### DETAILED DESCRIPTION

Technical solutions in some embodiments of the present disclosure will be described clearly and completely below with reference to the accompanying drawings. However, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as an open and inclusive meaning, i.e., “including, but not limited to”. In the description of the specification, the terms such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representation of the above terms does not necessarily refer to the same embodiment(s) or example(s). In addition, the specific features, structures, materials or characteristics may be included in any one or more embodiments or examples in any suitable manner.

Hereinafter, the terms such as “first” and “second” are used for descriptive purposes only, but are not to be construed as indicating or implying the relative importance or implicitly indicating the number of indicated technical features. Thus, the features defined with “first” and “second” may explicitly or implicitly include one or more of the features. In the description of the embodiments of the present disclosure, the term “a plurality of/the plurality of” means two or more unless otherwise specified.

The phrase “at least one of A, B and C” has a same meaning as the phrase “at least one of A, B or C”, and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

The phrase “applicable to” or “configured to” as used herein indicates an open and inclusive expression, which does not exclude devices that are applicable to or configured to perform additional tasks or steps.

Some embodiments of the present disclosure provide a deoxyribonucleic acid (DNA) library construction kit. The DNA library construction kit includes: a DNA end repair kit, a DNA adapter ligation kit, a DNA amplification kit, etc.

DNA is a long-chain polymer, units of which are four deoxynucleotides, i.e., adenine deoxynucleotide (dAMP), thymidine deoxynucleotide (dTMP), cytosine Pyrimidine deoxynucleotide (dCMP) and Guanine deoxynucleotide (dGMP).

A kit is a box used to contain chemical reagents for detection of chemical components, drug residues, virus types, etc. Of course, those having ordinary skills in the art will understand that, in order to contain the chemical reagents, the kit may also be some other container such as a tube.

Some embodiments of the present disclosure provide a DNA end repair kit. The DNA end repair kit includes a DNA end repair reagent. The DNA end repair reagent may include: a DNA end repair combinatorial enzyme, deoxy-ribonucleoside triphosphate (dNTP, including deoxyadenosine triphosphate (dATP)), a polyethylene glycol (PEG), a buffer, etc.

The DNA end repair combinatorial enzyme is used to combine with a DNA fragment having sticky ends (5′ protruding ends or 3′ protruding ends of the DNA fragment) to catalyze a 5′-3′ synthesis reaction and a 3′-5′ excision reaction of the DNA fragment to fill overhangs of the 5′ end and/or blunt overhangs of the 3′ end, so that a complete double-stranded DNA is formed, which is conductive to adenine (A) addition to 3′ end and a subsequent adapter ligation reaction.

Nicks of two single strands of DNA cut by a restriction enzyme have several protruding nucleotides which are just complementary in pairs. Such nicks provide sticky ends. That is, the restriction enzyme cuts the DNA at different positions on the two single strands of the DNA, and ends of a resulting double-stranded DNA are not flush, but one single strand is longer.

The dNTP is an abbreviation of deoxyribonucleoside triphosphate, which is a collective name of dATP (also referred to as 3′-deoxyadenosine), 2′-deoxyaguanosine-5′-triphosphate trisodium salt (dGTP, also referred to as deoxyguanosine triphosphate trisodium), deoxythymidine triphosphate (dTTP) and deoxycytidine triphosphate (dCTP), etc., where N refers to a nitrogenous base, and is a variable which represents one of A, T, G, C, etc. The dNTP, which is a raw material for synthesizing DNA, is a raw material for end repair here. The dNTP serves as a raw material for A addition to ends.

The PEG is used to occupy a certain space instead of water molecules, so as to increase a probability that the DNA fragment contacts with the DNA end repair combinatorial enzyme, which speeds up an enzymatic reaction and improves a repair rate.

The buffer is used to adjust pH of an entire DNA end repair reaction system. For example, the buffer may keep the pH of the entire DNA end repair reaction system between 7.0 and 8.5, inclusive.

In some embodiments, the DNA end repair combinatorial enzyme includes: an enzyme I having a 5′-3′ DNA polymerase activity and a 3′-5′ DNA exonuclease activity, an enzyme II capable of adding A to a 3′ end of DNA in a presence of dNTP, and an enzyme III capable of phosphorylating a 5′ end of the DNA.

In some embodiments, the enzyme I may include a T4 DNA polymerase and a Klenow fragment.

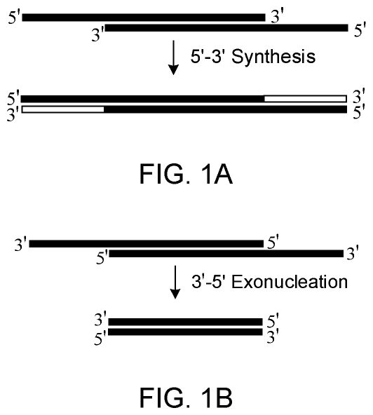

The T4 DNA polymerase has both the 5′-3′ DNA polymerase activity and the 3′-5′ DNA exonuclease activity. The 5′-3′ DNA polymerase activity of T4 DNA polymerase can catalyze a 5′-3′ synthesis reaction of DNA to fill overhangs of a 5′ end. The 3′-5′ DNA exonuclease activity of T4 DNA polymerase can catalyze a 3′-5′ exonucleation reaction of DNA to blunt overhangs of a 3′ end. FIG. 1A shows an example where overhangs of 5′ ends are filled through the 5′-3′ synthesis reaction of DNA. FIG. 1B shows an example where overhangs of 3′ ends are blunt through the 3′-5′ exonucleation reaction. The Klenow fragment (also referred to as a Klenow enzyme) is a fragment with a C-terminal, 605 amino acid residues which is generated by partial hydrolysis of a DNA polymerase I (E. coli) by trypsin or subtilisin. The Klenow fragment retains the 5′-3′ DNA polymerase activity and the 3′-5′ DNA exonuclease activity of the DNA polymerase I. Action mechanisms of the 5′-3′ DNA polymerase activity and 3′-5′ DNA exonuclease activity of the Klenow fragment are same as action mechanisms of the 5′-3′ DNA polymerase activity and 3′-5′ DNA exonuclease activity of the T4 DNA polymerase.

In some embodiments, a molecular weight of the Klenow fragment is 76 kDa.

In some embodiments, an amino acid sequence of the Klenow fragment is as shown in a sequence 3 in a sequence listing. The sequence 3 is as follows:

VISYDNYVTILDEETLKAWIAKLEKAPVFAFDTETDSLDNISANLVGLS
FAIEPGVAAYIPVAHDYLDAPDQISRERALELLKPLLEDEKALKVGQNL
KYDRGILANYGIELRGIAFDTMLESYILNSVAGRHDMDSLAERWLKHKT
ITFEEIAGKGKNQLTFNQIALEEAGRYAAEDADVTLQLHLKMWPDLQKH
KGPLNVFENIEMPLVPVLSRIERNGVKIDPKVLHNHSEELTLRLAELEK
KAHEIAGEEFNLSSTKQLQTILFEKQGIKPLKKTPGGAPSTSEEVLEEL
ALDYPLPKVILEYRGLAKLKSTYTDKLPLMINPKTGRVHTSYHQAVTAT
GRLSSTDPNLQNIPVRNEEGRRIRQAFIAPEDYVIVSADYSQIELRIMA
HLSRDKGLLTAFAEGKDIHRATAAEVFGLPLETVTSEQRRSAKAINFGL
IYGMSAFGLARQLNIPRKEAQKYMDLYFERYPGVLEYMERTRAQAKEQG
YVETLDGRRLYLPDIKSSNGARRAAAERAAINAPMQGTAADIIKRAMIA
VDAWLQAEQPRVRMIMQVHDELVFEVHKDDVDAVAKQIHQLMENCTRLD
VPLLVEVGSGENWDQAH.

In some other embodiments, the enzyme I may include the T4 DNA polymerase and a mutant of the Klenow fragment.

The mutant of the Klenow fragment is obtained by truncating the Klenow fragment, and is a recombinase expressed by Escherichia coli. After point mutation recombination, both the 5′-3′ DNA polymerase activity and the 3′-5′ DNA exonuclease activity of the enzyme are improved.

For example, the mutant of the Klenow fragment is obtained by mutating a 442nd phenylalanine in the Klenow fragment to a tyrosine. A molecular weight of the mutant of the Klenow fragment is 68.2 kDa.

In some embodiments, an amino acid sequence of the mutant of the Klenow fragment is as shown in a sequence 2 in the sequence listing. The sequence 2 is as follows:

VISYDNYVTILDEETLKAWIAKLEKAPVFAFDTETDSLDNISANLVGLS
FAIEPGVAAYIPVAHDYLDAPDQISRERALELLKPLLEDEKALKVGQNL
KYDRGILANYGIELRGIAFDTMLESYILNSVAGRHDMDSLAERWLKHKT
ITFEEIAGKGKNQLTFNQIALEEAGRYAAEDADVTLQLHLKMWPDLQKH
KGPLNVFENIEMPLVPVLSRIERNGVKIDPKVLHNHSEELTLRLAELEK
KAHEIAGEEFNLSSTKQLQTILFEKQGIKPLKKTPGGAPSTSEEVLEEL
ALDYPLPKVILEYRGLAKLKSTYTDKLPLMINPKTGRVHTSYHQAVTAT
GRLSSTDPNLQNIPVRNEEGRRIRQAFIAPEDYVIVSADYSQIELRIMA
HLSRDKGLLTAFAEGKDIHRATAAEVFGLPLETVTSEQRRSAKAINYGL
IYGMSAFGLARQLNIPRKEAQKYMDLYFERYPGVLEYMERTRAQAKEQG
YVETLDGRRLYLPDIKSSNGARRAAAERAAINAPMQGTAADIIKRAMIA
VDAWLQAEQPRVRMIMQVHDELVFEVHKDDVDAVAKQIHQLMENCTRLD
VPLLVEVGSGENWDQAH.

In some embodiments, the enzyme II may include a Taq DNA polymerase, and the enzyme III may include a T4 polynucleotide kinase (T4 PNK).

In some embodiments, a concentration of the T4 DNA polymerase in the DNA end repair reagent is in a range of 0.02 U/μL to 0.1 U/μL, inclusive. For example, the concentration may be 0.02 U/μL, 0.03 U/μL, 0.04 U/μL, 0.05 U/μL, 0.06 U/μL, 0.07 U/μL, 0.08 U/μL, 0.09 U/μL or 0.1 U/μL. A concentration of the T4 PNK in the DNA end repair reagent is in a range of 0.05 U/μL to 0.15 U/μL, inclusive. For example, the concentration may be 0.05 U/μL, 0.06 U/μL, 0.07 U/μL, 0.08 U/μL, 0.09 U/μL, 0.1 U/μL, 0.11 U/μL, 0.12 U/μL, 0.13 U/μL, 0.14 U/μL or 0.15 U/μL. A concentration of the Taq DNA polymerase in the DNA end repair reagent is in a range of 0.02 U/μL to 0.15 U/μL, inclusive. For example, the concentration may be 0.02 U/μL, 0.03 U/μL, 0.04 U/μL, 0.05 U/μL, 0.06 U/μL, 0.07 U/μL, 0.08 U/μL, 0.09 U/μL, 0.1 U/μL, 0.11 U/μL, 0.12 U/μL, 0.13 U/μL, 0.14 U/μL or 0.15 U/μL.

An enzyme activity, i.e., an amount of an enzyme, is expressed in enzyme activity unit (U). In 1961, the Enzymology Committee of the International Society of Biochemistry proposed to use a unified “international unit (IU)” to express the enzyme activity, which is stipulated as: under the optimum conditions (25° C.), an amount of the enzyme required to convert 1 micromol (μmol) of a substrate per minute into a product is defined as an activity unit. That is, 1 IU=1 μmol/min. That is, the amount of the enzyme may be expressed in units (U/g or U/ml) of enzyme activity per gram or per milliliter of an enzyme preparation, where U is an abbreviation of IU.

In some embodiments, in a case where the enzyme I includes the Klenow fragment, a concentration of the Klenow fragment in the DNA end repair reagent is in a range of 0.02 U/μL to 0.15 U/μL, inclusive. For example, the concentration may be 0.02 U/μL, 0.03 U/μL, 0.04 U/μL, 0.05 U/μL, 0.06 U/μL, 0.07 U/μL, 0.08 U/μL, 0.09 U/μL, 0.1 U/μL, 0.11 U/μL, 0.12 U/μL, 0.13 U/μL, 0.14 U/μL or 0.15 U/μL.

In some embodiments, in a case where the enzyme I includes the mutant of the Klenow fragment, a concentration of the mutant of the Klenow fragment in the DNA end repair reagent is in a range of 0.02 U/μL to 0.15 U/μL, inclusive. For example, the concentration may be 0.02 U/μL, 0.03 U/μL, 0.04 U/μL, 0.05 U/μL, 0.06 U/μL, 0.07 U/μL, 0.08 U/μL, 0.09 U/μL, 0.1 U/μL, 0.11 U/μL, 0.12 U/μL, 0.13 U/μL, 0.14 U/μL or 0.15 U/μL.

In some embodiments, the PEG is one or more selected from a PEG-4000, a PEG-6000, and a PEG-8000.

The PEG-4000 refers to a PEG with a molecular weight of 4000, the PEG-6000 refers to a PEG with a molecular weight of 6000, and the PEG-8000 refers to a PEG with molecular weight of 8000.

By selecting the molecular weight of the PEG, target DNA fragments (e.g., DNA fragments to be sequenced) may be effectively contacted with the DNA end repair combinatorial enzyme, while other DNA fragments (e.g., molecular weights of these DNA fragments being greater than or less than that of the target DNA fragments) are kept away from the DNA end repair combinatorial enzyme, so that enzymatic effects may be improved, and then a subsequent library conversion efficiency may be improved.

In some embodiments, the PEG is the PEG-4000. It will be found through experiments that, in a case where the molecular weight of the selected PEG is 4000, the enzymatic effects may be improved to a greatest extent.

In some embodiments, a mass percentage of the PEG in the DNA end repair reagent is in a range of 8% to 25%, inclusive. For example, the mass percentage may be 8%, 9%, 10%, 11%, 12%, 13%, 14%, 15%, 16%, 17%, 18%, 19%, 20%, 21%, 22%, 23%, 24% or 25%.

In some embodiments, the DNA end repair reagent further includes: a single strand DNA-binding protein (SSB).

The SBB may also be referred to as a DNA-binding protein, which is necessary for DNA replication. After the DNA unwinds, DNA molecules have a tendency to combine into double strands as long as bases are paired. The binding of the SSB occurs in a single-stranded region produced when a helicase advances in a direction of a replication fork, which may prevent newly formed single-stranded DNA from re-pairing to form double-stranded DNA or a protein that is capable of degrading by nucleases. In addition, after the SSB bind to the single-stranded DNA, the single-stranded DNA is kept in an extending state without bends and knots, which is beneficial to use the single-stranded DNA as a template.

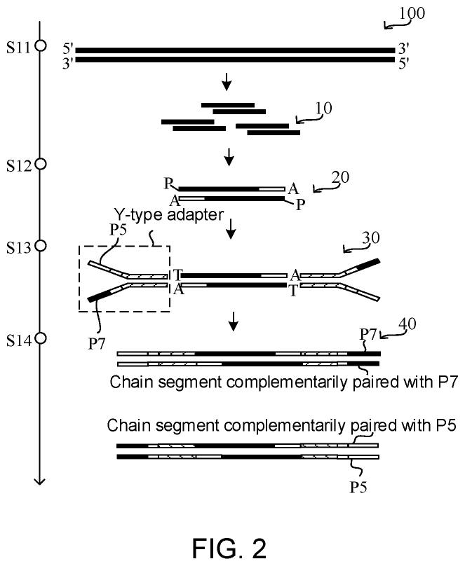

In these embodiments, by adding the SSB to the DNA end repair reagent and using a property of the SSB to bind to the single-stranded DNA, the SSB may bind to the sticky ends in the process of end repair and A addition. It may be possible to make the sticky ends of the DNA fragments (e.g., first DNA fragments 10 shown in FIG. 2) in the extending state without bend and knot, which may prevent a DNA super-secondary structure (e.g., a hairpin structure) from being formed. In this way, it may be conductive to binding the DNA end repair combinatorial enzyme, such as the enzyme I, to the sticky ends of the DNA fragments to promote the enzymatic reaction; and the SSB will fall off when a newly formed DNA strand binds to a sticky end at a position where the SSB binds, which is beneficial to repair the sticky ends of the DNA fragments, improve a repair efficiency, and prevent a loss caused by a large number of DNA fragments that fail to form complete double-stranded DNA. Therefore, a library yield may be increased, and the library conversion efficiency may be improved. Moreover, in an initial stage of a whole process of the enzymatic reaction, by binding the SSB to the sticky ends of the DNA fragments, it may be possible to protect the sticky ends of the DNA fragments from undergoing an enzymatic hydrolysis reaction, so that the DNA fragments may be kept intact. Therefore, defects, such as missing, in the DNA fragments to be sequenced during a library construction process may be avoided, and the constructed library may meet requirements for using high-throughput sequencing and back-end targeted sequencing.

Considering an example where a number of bases in a sticky end of the DNA fragment on a single side is in a range of 10 to 20, inclusive, as long as a single-stranded binding protein specifically binds to some bases (e.g., 8 to 16 bases) of the bases, the sticky end of the DNA fragment may be protected, and be kept in the extending state.

In some embodiments, the SSB may be a T4 gene 32 protein. The T4 gene 32 protein exists in a form of a tetramer with a molecular weight of 33 KDa. The T4 gene 32 protein may coordinately bind to and stabilize a transiently formed DNA single-stranded region, and play a role of making the sticky ends of the DNA fragments in the extending state. In addition, as shown from studies, the T4 gene 32 protein may also enhance an activity of the T4 DNA polymerase, which may speed up DNA end repair.

In some embodiments, an amino acid sequence of the SSB is as shown in sequence 1 in the sequence listing. The sequence 1 is as follows:

MFKRKSTAELAAQMAKLNGNKGFSSEDKGEWKLKLDNAGNGQAVIRFLP
SKNDEQAPFAILVNHGFKKNGKWYIETCSSTHGDYDSCPVCQYISKNDL
YNTDNKEYSLVKRKTSYWANILVVKDPAAPENEGKVFKYRFGKKIWDKI
NAMIAVDVEMGETPVDVTCPWEGANFVLKVKQVSGFSNYDESKFLNQSA
IPNIDDESFQKELFEQMVDLSEMTSKDKFKSFEELNTKFGQVMGTAVMG
GAAATAAKKADKVADDLDAFNVDDFNTKTEDDFMSSSSGSSSSADDTDL
DDLLNDL.

In some embodiments, a size of the DNA fragment may be in a range of 150 bp to 200 bp, inclusive. A unit of the size of the DNA fragment is measured in base pairs. The commonly used units are base pair (bp), kilobase pair (Kbp) and megabase pair (Mbp). The description here takes an example where the unit of the size of the DNA fragment is bp. That is, a number of base pairs included in the obtained DNA fragment (including the sticky ends) is in a range of 150 to 200, inclusive.

In some embodiments, a concentration of the SSB in the DNA end repair reagent is in a range of 0.5 μg/μL to 2 μg/μL, inclusive. For example, the concentration may be 0.5 μg/μL, 0.6 μg/μL, 0.7 μg/μL, 0.8 μg/μL, 0.9 μg/μL, 1 μg/μL, 1.1 μg/μL, 1.2 μg/μL, 1.3 μg/μL, 1.4 μg/μL, 1.5 μg/μL, 1.6 μg/μL, 1.7 μg/μL, 1.8 μg/μL, 1.9 μg/μL or 2.0 μg/μL.

By limiting the concentration of the SSB in the DNA end repair reagent within the range, when used, the DNA end repair reaction system may be used directly by configuring the DNA end repair reaction system, e.g., diluting the DNA end repair reagent by a certain factor. In addition, it is proved by experiments that, the library yield and the library conversion efficiency may effectively be improved by limiting the concentration of the SSB in the DNA end repair reagent within the range during use.

It will be noted that, the dNTP, the dATP, the PEG, the buffer, the T4 DNA polymerase, the Klenow fragment, the mutant of the Klenow fragment and the SSB mentioned above are all commercially available.

It will also be noted that, the above description only shows the example where the enzyme I includes the T4 DNA polymerase and the Klenow fragment and the example where the enzyme I include the T4 DNA polymerase and the mutant of the Klenow fragment. Those having ordinary skills in the art will understand that, the enzyme I may only include any one of the T4 DNA polymerase, the Klenow fragment and the mutant of the Klenow fragment; or in some other embodiments, the enzyme I may include both the Klenow fragment and the mutant of the Klenow fragment.

Some embodiments of the present disclosure provide a DNA adapter ligation kit. The DNA adapter ligation kit includes a DNA adapter ligation reagent.

In some embodiments, the DNA adapter ligation reagent includes: a DNA ligase, sequencing adapters, a buffer, a PEG, etc.

The DNA ligase is used to promote a ligation of a sequencing adapter and an end-repaired DNA fragment. For example, the sequencing adapter may be a Y-type adapter. The buffer provides a stable pH environment for an adapter ligation reaction. The PEG has same function as the PEG included in the DNA end repair reagent, and may also increase a contact probability that the target DNA fragments contact with the DNA ligase, which improves a yield of adapter ligation.

In some embodiments, the PEG is a PEG-4000. The PEG-4000 refers to a PEG with a molecular weight of 4000.

Compared with related art where a PEG is a PEG-8000, such a design may effectively enable the target DNA fragments (e.g., the DNA fragments to be sequenced) to contact with the DNA ligase, and enable other DNA fragments (e.g., the molecular weights of these DNA fragments being greater than or less than that of the target DNA fragments) to be kept away from the DNA ligase, which may improve the adapter ligation yield.

In some embodiments, a concentration of the PEG in the DNA adapter ligation reagent is in a range of 8% to 25%, inclusive. For example, the concentration may be 8%, 9%, 10%, 11%, 12%, 13%, 14%, 15%, 16%, 17%, 18%, 19%, 20%, 21%, 22%, 23%, 24% or 25%.

In some embodiments, a concentration of the DNA ligase in the DNA adapter ligation reagent is in a range of 0.3 U/μL to 3 U/μL, inclusive; and the buffer keeps pH of the DNA adapter ligation reaction system in a range of 7.0 to 8.5, inclusive.

Some embodiments of the present disclosure provide a method for constructing a DNA library. As shown in FIG. 2, the method includes the following steps.

In a step S11, a genomic DNA 100 is fragmented to obtain first DNA fragments 10.

The genomic DNA 100 may be a genomic DNA of any one of animals, plants, viruses, etc.

Fragmenting the genomic DNA 100 may include:
fragmenting the genomic DNA 100 by mechanical means or enzymatic hydrolysis.

In a case where the genomic DNA 100 is fragmented by mechanical means, an ultrasonic crusher may be used to perform ultrasonic fragmentation processing on the genomic DNA 100. In a case where the genomic DNA is fragmented by enzymatic hydrolysis, a non-specific nuclease may be used to fragment the genomic DNA 100 randomly.

The genomic DNA 100 may be obtained by DNA extraction or by commercial means, which is not limited here.

In some embodiments, a size of the first DNA fragment 10 may be in a range of 150 bp 200 bp, inclusive.

Here, it will be noted that, both ends of most of the fragmented DNA by mechanical means and enzymatic hydrolysis will have a sticky end of 5′ overhangs or 3′ overhangs. Nicks of two cut single strands of DNA have several protruding nucleotides which are just complementary in pairs. Such nicks provide sticky ends. That is, the DNA is cut at different positions on the two single strands of DNA, and ends of a resulting double-stranded DNA are not flush, but one strand is longer.

In a step S12, the first DNA fragments 10 are treated with the DNA end repair reagent to obtain second DNA fragments 20. The second DNA fragment 20 is a fragment with flush ends which is phosphorylated at 5′ ends and with A at 3′ ends. A treatment condition is: firstly treating at 15° C. to 25° C. for 10 min to 20 min, and then treating at 60° C. to 70° C. for 10 min to 20 min.

In order to sequence a fragmented DNA fragment, there need to repair the ends thereof first, so that the ligation requirements of the sequencing adapter are met.

Treating the first DNA fragments 10 by using the DNA end repair reagent may include the following step.

As shown in FIGS. 1A and 1B, the DNA end repair combinatorial enzyme included in the DNA end repair reagent is used to catalyze a 5′-3′ synthesis reaction and a 3′-5′ exosection reaction of the first DNA fragment 10, so that overhangs of the 5′ end are filled and/or overhangs of the 3′ end are blunted to form a complete double-stranded DNA, and the double-stranded DNA undergoes a phosphorylation reaction at the 5′ end and an A addition reaction at the 3′ end.

During this process, at 15° C. to 25° C., the enzyme I and the enzyme III in the DNA end-repair combinatorial enzyme play a catalytic role to perform end repair and blunting; and at 60° C. to 70° C., the enzyme I and the enzyme III in the DNA end-repair combinatorial enzyme denature, so that the enzyme II plays a catalytic role of adding A to the end.

For example, the T4 DNA polymerase and the Klenow fragment have the 5′-3′ DNA polymerase activity and the 3′-5′ exonuclease activity, and may fill the 5′ sticky end of the DNA fragment and cut off the protruding 3′ sticky end of the DNA fragment in a presence of dNTP at 15° C. to 25° C. The T4 PNK (the T4 polynucleotide kinase) may add a phosphoric acid group at the 5′ end. The Taq DNA polymerase may add an A at the 3′ end in a presence of ATP at 60° C. to 70° C. Through the above process, the end repair and the A addition of the DNA fragment (i.e., the first DNA fragment 10) obtained by cutting the genomic DNA may be completed.

In addition, since the DNA end repair reagent further includes the SSB, the SSB may bind to the sticky ends in the process of end repair and A addition. It may be possible to make the sticky ends of the DNA fragments (i.e., the first DNA fragments 10) in the extending state without bend and knot, which may prevent a DNA super-secondary structure (e.g., a hairpin structure) from being formed. In this way, it may be conductive to binding the DNA end repair combinatorial enzyme, such as the enzyme I, to the sticky ends of the DNA fragments to promote the enzymatic reaction; and when the newly formed DNA strand binds to the sticky end at the position where the SSB binds, the SSB will fall off, which is beneficial to repair the sticky ends of the DNA fragments, improve the repair efficiency, and prevent the loss caused by a large number of DNA fragments that fail to form complete double-stranded DNA. Therefore, the library yield may be increased, and the library conversion efficiency may be improved. Moreover, in the initial stage of the whole process of the enzymatic reaction, by binding the SSB to the sticky ends of the DNA fragments, it may be possible to protect the sticky ends of the DNA fragments from undergoing an enzymatic hydrolysis reaction, so that the DNA fragments may be kept intact. Therefore, the defects, such as missing, in the DNA fragments to be sequenced during the library construction process may be avoided, and the constructed library may meet requirements for using high-throughput sequencing and back-end targeted sequencing.

In a step S13, sequencing adapters are ligated to the second DNA fragments 20 to obtain adapter ligation products 30.

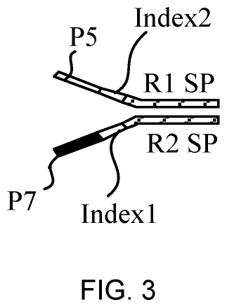

For example, the sequencing adapter may be a Y-type adapter. As shown in FIG. 3, the Y-type adapter may include a P5/P7 sequence, an Index sequence and an R1 SP/R2 SP sequence. The P5/P7 sequence can be complementary and identical to the P5/P7 sequence on a sequencing chip, so that a fragment (i.e., the second DNA fragment 20) to be sequenced may be fixed on a Flowcell for bridge-type polymerase chain reaction (PCR) amplification. The “Index” is also referred to as barcode, whose aim is to add specific tags used to distinguish different library samples when libraries are mixed and sequenced. The R1 SP/R2 SP is a region where Read1 and Read2 sequencing primers bind, and can carry out base extension due to an action of dNTP and the DNA polymerase.

The Y-shaped adapter ensures that both ends of each single sequence has different sequencing primers, so as to form a library with two ends having different nucleotide sequences (P5/P7) by a subsequent PCR amplification.

Depend on different positions of the Indexes, sequencing adapters may be classified into single-ended Index adapters and double-ended Index adapters. The single-ended Index adapter only has an Index sequence at a P7 end, and the double-ended Index adapter has an Index sequence at each of a P5 end and the P7 end. FIG. 3 shows an example where the sequencing adapter is the double-ended Index adapter.

Ligating the sequencing adapters to the second DNA fragments 20 may include the following step.
treating the second DNA fragments 20 with the DNA adapter ligation reagent described above, so as to ligate sequencing adapters to the second DNA fragments 20.

The DNA ligase included in the DNA adapter ligation reagent is used to cause the second DNA fragments 20 to react with the sequencing adapters.

In the embodiments of the present disclosure, since the DNA adapter ligation reagent includes the PEG-4000, it will be found through experiments that, compared with a PEG-8000 in the related art, the target DNA fragments (e.g., the DNA fragments to be sequenced, i.e., the DNA fragments whose sizes are in the range of 150 bp to 200 bp) may be effectively contacted with the DNA ligase, while other DNA fragments (e.g., the molecular weights of these DNA fragments being greater than or less than the target DNA fragments) are kept away from the DNA ligase, so that the enzymatic effects may be improved, and then the subsequent library conversion efficiency may be improved.

The second DNA fragments 20 react with the sequencing adapters at a temperature of 20° C. to 25° C. for 15 min to 20 min.

In a step S14, the adapter ligation products 30 are purified, and the purified products are enriched to obtain a DNA library.

Purifying the adapter ligation products 30 may include:

absorbing the adapter ligation products 30 by magnetic beads, so as to remove the enzymes, salt ions and residual sequencing adapters in the reaction system.

A surface of the magnetic bead has a linking group, and the linking group may be combined with the adapter ligation product 30 by electrostatic interaction, so as to realize an adsorption of the magnetic bead to the adapter ligation product 30.

Enriching the purified products may be include:

enriching the purified products by PCR amplification method.

The amplification method includes three reaction steps of denaturation, annealing and extension. For example, the purified products may be heated to about 95° C. for a certain period of time (e.g., 3 min) to dissociate each purified product into two single strands; and then the temperature is raised to 98° C. for a certain period of time (e.g., 20 s) to ensure that the purified products are completely denatured (i.e., each purified product is denatured into two single strands); and then the temperature is lowered to about 60° C. for a certain period of time (e.g., 30 s), two primers included in a primer pair respectively bind to the two single strands obtained by dissociation through complementary base pairing (one primer in the primer pair (e.g., a primer 1) is a fragment in a chain segment complementarily paired with P7 in FIG. 2, and the other primer (e.g., a primer 2) is a fragment in the chain segment complementarily paired with P5 in FIG. 2); and then, the temperature is raised to 72° C. for a certain period of time (e.g., 30 s), so that a combination of each single-stranded DNA and a respective primer synthesizes a replicating strand complementary to the single-stranded DNA due to an action of the DNA polymerase, and then a double-stranded DNA is formed. By repeating this cycle for 5 to 10 times, enrichment products 40 can be obtained.

In addition, in order to obtain enrichment products with high purity, the method for constructing a DNA library further includes: purifying the enrichment products 40 by using magnetic beads.

The DNA fragments may be purified by magnetic bead method.

In order to objectively evaluate technical effects of the embodiments of the present disclosure, detailed exemplary description of embodiments of the present application is given through the following comparative examples and experimental examples.

### Comparative Example 1

A method for constructing a DNA library in Comparative Example 1 is as follows.
In a step 1), genomic DNA is fragmented. This step includes: adding 1 ng to 100 ng (e.g., 1 ng, 10 ng, 50 ng, and 100 ng) of a sample to 0.6 mL PCR tubes, respectively; making up to 50 μL with a Tris-EDTA (TE) buffer solution; mixing well and centrifuging; breaking the samples with a Bioruptor ultrasonic crusher for 20 cycles to obtain DNA fragments, the ultrasonic crusher being turned on for 30 s and turned off for 30 s in each cycle; purifying the DNA fragments with AMpure XP magnetic beads to obtain DNA fragments 10_a with sizes of 150 bp to 200 bp after ultrasonication.
In a step 2), a DNA end repair reagent is prepared. Specific components and concentrations are shown in Table 1 below.

TABLE 1

| Component | Concentration | |
|---|---|---|
| T4 DNA polymerase | 0.05 | U/μL |
| Klenow Fragment | 0.1 | U/μL |
| T4 PNK | 1 | U/μL |
| Taq DNA polymerase | 0.1 | U/μL |
| 1*Buffer solution (e.g., Tris(hydroxymethyl)methyl | 10 | mM |
| aminomethane (TRIS) - HCl) | | |
| dNTP | 1 | mM |
| Excess dATP | 1 | mM |

| | |
|---|---|
| PEG-4000 | 15% |
| Water | / |

In a step 3), the DNA end repair reagent shown in Table 1 is used to perform end repair and A addition on the DNA fragments 10_a with the sizes of 150 bp to 200 bp. 15 μL of the DNA end repair reagent may be mixed with 50 μL of the DNA fragments 10_a; and the two are put in a small tube; the tube are put in a heater to be kept at 20° C. for 15 min and then at 65° C. for 15 min, so as to carry out an reaction to finally obtain end repair products 20_a.
In a step 4), an adapter ligation reaction system is prepared. Specific components and volumes are shown in Table 2 below. The system is kept at 20° C. for 15 min to obtain adapter ligation products 30_a.

TABLE 2

| Component | Volume (μL) |
|---|---|
| End repair product | 65 |
| 1*Buffer (such as Tris(hydroxymethyl)methyl | 10 |
| aminomethane (TRIS) - HCl) (10 mM) | |
| DNA ligase | 5 |
| PEG-4000 (15%) | 10 |
| Sequencing adapter | 3 |
| Water | 7 |

In a step 5), 88 μL of magnetic beads are added to the adapter ligation products 30_a; the two are mixed well and stand at room temperature for 5 min, and are placed on a magnetic frame for about 5 min to make the magnetic beads completely adsorb the adapter ligation products 30_a and make the solution clarified; a supernatant is carefully removed; 200 μL of freshly prepared 80% ethanol is added to rinse a remainder, the mixture stands at room temperature for 30 s to 60 s, and then a supernatant is carefully removed; the previous step is repeated once; after the magnetic beads are dry, 22 μL of ultrapure water is added for elution, the mixture are placed on the magnetic frame to stand at room temperature for 3 min; and after the solution becomes clear, 20 uL of a supernatant, i.e., the purified adapter ligation products 30_a, are pipetted.
In a step 6), a PCR enrichment reaction system is prepared. Specific components and volumes are shown in Table 3 below. The 20 μL of purified adapter ligation products 30_a and 30 μL of PCR enrichment reagent are put in a tube, and heated to about 95° C. for 3 min first, further to 98° C. for 20 s, then cooled down to about 60° C. for 30 s, and finally heated to 72° C. for 30 s to complete a cycle. Such a cycle is repeated 5 to 10 times to obtain enrichment products 40_a.

TABLE 3

| Component | Volume (μL) |
|---|---|
| Purified adapter ligation product | 20 |
| Primer Pair mixed liquor | 5 |
| 2*Hot start DNA polymerase mixed | 25 |
| liquor | |

In a step 7), 60 μL of magnetic beads are added to the enrichment products 40_a; the two are mixed well, stand at room temperature for 5 min, and are placed on the magnetic frame for about 5 min to make the magnetic beads completely adsorb the enrichment products 40_a and make the solution clarified; a supernatant is carefully removed; 200 μL of freshly prepared 80% ethanol is added to rinse a remainder, the mixture stands at room temperature for 30 s to 60 s, and then a supernatant is carefully removed; the previous step is repeated once; after the magnetic beads are dry, 22 μL of ultrapure water is added for elution, the mixture are placed on the magnetic frame to stand at room temperature for 3 min; and after the solution becomes clear, 20 μL of a supernatant, i.e., purified enrichment products 40_a, are pipetted.

### Experimental Example 1

A method for constructing a DNA library of Experimental Example 1 is substantially same as that of Comparative Example 1. A difference is that, specific components and concentrations of a prepared DNA end repair reagent in a step 2) of Experimental Example 1 are as shown in Table 4 below. That is, compared with Comparative Example 1, the DNA end repair reagent in Experimental Example 1 further includes a SSB with a concentration of 1 μg/μL, and final purified enrichment products are represented as enrichment products 40_b.

TABLE 4

| Component | Concentration | |
|---|---|---|
| T4 DNA polymerase | 0.05 | U/μL |
| Klenow Fragment | 0.1 | U/μL |
| T4 PNK | 1 | U/μL |
| Taq DNA polymerase | 0.1 | U/μL |
| 1*Buffer solution (e.g., Tris(hydroxymethyl)methyl | 10 | mM |
| aminomethane (TRIS) - HCl) | | |
| SSB | 1 | μg/μL |
| dNTP | 1 | mM |
| Excess dATP | 1 | mM |

| | |
|---|---|
| PEG-4000 | 15% |
| Water | / |

### Comparative Example 2

A method for constructing a DNA library of Comparative Example 2 is substantially same as that of Comparative Example 1. A difference is that, specific components and concentrations of a prepared DNA end repair reagent in a step 2) of Comparative Example 2 are as shown in Table 5 below. That is, compared with Comparative Example 1, the DNA end repair reagent in Comparative Example 2 adopts a mutant of Klenow fragment, and final purified enrichment products are represented as enrichment products 40_c.

TABLE 5

| Component | Concentration | |
|---|---|---|
| T4 DNA polymerase | 0.05 | U/μL |
| Mutant of Klenow fragment | 0.1 | U/μL |
| T4 PNK | 1 | U/μL |
| Taq DNA polymerase | 0.1 | U/μL |
| 1*Buffer solution (e.g., Tris(hydroxymethyl)methyl | 10 | mM |
| aminomethane (TRIS) - HCl) | | |
| dNTP | 1 | mM |
| Excess dATP | 1 | mM |

| | |
|---|---|
| PEG-4000 | 15% |
| Water | / |

### Experimental Example 2

A method for constructing a DNA library of Experimental example 2 is substantially same as that of Comparative Example 2. A difference is that, specific components and concentrations of a prepared DNA end repair reagent in a step 2) of Experimental example 2 are as shown in Table 6 below. That is, compared with Comparative Example 2, a DNA end repair reagent in Experimental Example 2 further includes a SSB with a concentration of 1 μg/μL, and final purified enrichment products are represented as enrichment products 40_d.

TABLE 6

| Component | Concentration | |
|---|---|---|
| T4 DNA polymerase | 0.05 | U/μL |
| Mutant of Klenow fragment | 0.1 | U/μL |
| T4 PNK | 1 | U/μL |
| Taq DNA polymerase | 0.1 | U/μL |
| 1*Buffer solution (e.g., Tris(hydroxymethyl)methyl | 10 | mM |
| aminomethane (TRIS) - HCl) | | |
| SSB | 1 | μg/μL |
| dNTP | 1 | mM |
| Excess dATP | 1 | mM |

| | |
|---|---|
| PEG-4000 | 15% |
| Water | / |

### Experimental Example 3

A method for constructing a DNA library of Experimental example 2 is substantially same as that of Comparative Example 2. A difference is that, specific components and concentrations of a prepared DNA end repair reagent in a step 2) of Experimental example 3 are as shown in Table 7 below, and final purified enrichment products are represented as enrichment products 40_e.

TABLE 7

| Component | Concentration | |
|---|---|---|
| T4 DNA polymerase | 0.02 | U/μL |
| Mutant of Klenow fragment | 0.02 | U/μL |
| T4 PNK | 0.5 | U/μL |
| Taq DNA polymerase | 0.02 | U/μL |
| 0.5*Buffer solution (e.g., Tris(hydroxymethyl)methyl | 5 | mM |
| aminomethane (TRIS) - HCl) | | |
| SSB | 2 | μg/μL |
| dNTP | 0.5 | mM |
| Excess dATP | 0.5 | mM |

| | |
|---|---|
| PEG-4000 | 8% |
| Water | / |

### Experimental Example 4

A method for constructing a DNA library of Experimental example 4 is substantially same as that of Experimental Example 2. A difference is that, specific components and concentrations of a prepared DNA end repair reagent in a step 2) of Experimental example 3 are as shown in Table 8 below, and final purified enrichment products are represented as enrichment products 40_f.

TABLE 8

| Component | Concentration | |
|---|---|---|
| T4 DNA polymerase | 0.1 | U/μL |
| Mutant of Klenow fragment | 0.15 | U/μL |
| T4 PNK | 2 | U/μL |
| Taq DNA polymerase | 0.15 | U/μL |
| 1.5*Buffer solution (e.g., Tris(hydroxymethyl)methyl | 15 | mM |
| aminomethane (TRIS) - HCl) | | |
| SSB | 0.5 | μg/μL |
| dNTP | 2 | mM |
| Excess dATP | 2 | mM |

| | |
|---|---|
| PEG-4000 | 25% |
| Water | / |

### Experimental Example 5

A method for constructing a DNA library of Experimental example 5 is substantially same as that of Experimental Example 2. A difference is that, specific components and concentrations of a prepared DNA end repair reagent in a step 2) of Experimental example 3 are as shown in Table 9 below, and final purified enrichment products are represented as enrichment products 40_g.

TABLE 9

| Component | Concentration | |
|---|---|---|
| T4 DNA polymerase | 0.08 | U/μL |
| Mutant of Klenow fragment | 0.1 | U/μL |
| T4 PNK | 0.8 | U/μL |
| Taq DNA polymerase | 0.08 | U/μL |
| 1.2*Buffer solution (e.g., Tris(hydroxymethyl)methyl | 12 | mM |
| aminomethane (TRIS) - HCl) | | |
| SSB | 1.2 | μg/μL |
| dNTP | 1.2 | mM |
| Excess dATP | 1.2 | mM |

| | |
|---|---|
| PEG-4000 | 18% |
| Water | / |

Appropriate amounts of enrichment products that are different in initial input amounts of the sample are taken as respective measurement samples. For example, for each of Comparative Examples 1 to 2 and Experimental Examples 1 to 4, 1 μL of respective purified liquid enrichment products with initial input amounts of the genomic DNA of 1 ng, 10 ng, 50 ng and 100 ng are taken as measurement samples. Qubit 4.0 Fluorometer is used to measure concentrations of the enrichment products in the measurement samples and calculate respective library yields, library fragment means, and library conversion efficiencies at different initial input amounts of the sample for Comparative Examples 1 to 2 and Experimental Examples 1 to 4. Specific data are as shown in Table 10 below.

TABLE 10

| | | Initial | | | Library |
|---|---|---|---|---|---|
| | | input | | Library | conver- |
| | Group name | amount of | Library | fragment | sion |
| Sample | of | sample | yield | mean | effi- |
| name | experiment | (ng) | (ng) | (bp) | ciency |
| Enrichment | Comparative | 1 | 350 | 303 | 10.1% |
| product 40_a | Example 1 | | | | |
| Enrichment | Experimental | 1 | 1200 | 305 | 34.6% |
| product 40_b | Example 1 | | | | |
| Enrichment | Comparative | 1 | 430 | 296 | 12.4% |
| product 40_c | Example 2 | | | | |
| Enrichment | Experimental | 1 | 1696 | 300 | 48.9% |
| product 40_d | Example 2 | | | | |
| Enrichment | Experimental | 1 | 1650 | 295 | 47.5% |
| product 40_e | Example 3 | | | | |
| Enrichment | Experimental | 1 | 740 | 298 | 21.3% |
| product 40_f | Example 4 | | | | |
| Enrichment | Experimental | 1 | 1700 | 286 | 49.0% |
| product 40_g | Example 5 | | | | |
| Enrichment | Comparative | 10 | 340 | 299 | 10.3% |
| product 40_a | Example 1 | | | | |
| Enrichment | Experimental | 10 | 1150 | 312 | 34.8% |
| product 40_b | Example 1 | | | | |
| Enrichment | Comparative | 10 | 425 | 305 | 12.9% |
| product 40_c | Example 2 | | | | |
| Enrichment | Experimental | 10 | 1600 | 300 | 48.4% |
| product 40_d | Example 2 | | | | |
| Enrichment | Experimental | 10 | 1499 | 287 | 45.3% |
| product 40_e | Example 3 | | | | |
| Enrichment | Experimental | 10 | 745 | 267 | 22.5% |
| product 40_f | Example 4 | | | | |
| Enrichment | Experimental | 10 | 1640 | 290 | 49.6% |
| product 40_g | Example 5 | | | | |
| Enrichment | Comparative | 50 | 340 | 297 | 12.0% |
| product 40_a | Example 1 | | | | |
| Enrichment | Experimental | 50 | 1130 | 313 | 39.9% |
| product 40_b | Example 1 | | | | |
| Enrichment | Comparative | 50 | 420 | 288 | 14.8% |
| product 40_c | Example 2 | | | | |
| Enrichment | Experimental | 50 | 1470 | 295 | 51.9% |
| product 40_d | Example 2 | | | | |
| Enrichment | Experimental | 50 | 1420 | 296 | 50.1% |
| product 40_e | Example 3 | | | | |
| Enrichment | Experimental | 50 | 730 | 305 | 25.8% |
| product 40_f | Example 4 | | | | |
| Enrichment | Experimental | 50 | 1520 | 285 | 53.6% |
| product 40_g | Example 5 | | | | |
| Enrichment | Comparative | 100 | 340 | 302 | 10.8% |
| product 40_a | Example 1 | | | | |
| Enrichment | Experimental | 100 | 1130 | 299 | 35.9% |
| product 40_b | Example 1 | | | | |
| Enrichment | Comparative | 100 | 420 | 287 | 13.3% |
| product 40_c | Example 2 | | | | |
| Enrichment | Experimental | 100 | 1560 | 305 | 49.5% |
| product 40_d | Example 2 | | | | |
| Enrichment | Experimental | 100 | 1540 | 312 | 48.9% |
| product 40_e | Example 3 | | | | |
| Enrichment | Experimental | 100 | 760 | 299 | 24.1% |
| product 40_f | Example 4 | | | | |
| Enrichment | Experimental | 100 | 1580 | 301 | 50.2% |
| product 40_g | Example 5 | | | | |

The library yield is obtained by multiplying a measured concentration of enrichment products by 50 μL. The library fragments mean is obtained by fluorescently labeling DNA fragments in a respective measurement sample, and measuring and averaging sizes of the DNA fragments in the measurement sample. The library conversion efficiency is equal to a ratio of a number of DNA fragments bound with sequencing adapters to a number of all DNA fragments in the measurement sample multiplied by 100%. The number of the DNA fragments bound with the sequencing adapters in the enrichment product are obtained by fluorescent quantitative PCR. The number of all DNA fragments in the enrichment product is equal to the concentration of the enrichment product multiplied by 50 μL and divided by the library fragment mean.

As will be seen from Table 10, compared with Comparative Example 1, by adding the SSB, a library yield and a library conversion efficiency of Experimental Example 1 are greatly improved, and a library fragment mean of Experimental Example 1 is not much different. Compared with Comparative Example 2, both library yields and library conversion efficiencies of Experimental Examples 2 to 5 are greatly improved, and similarly, library fragment means of Experimental Examples 2 to 5 are not much different. In addition, as will be seen from a comparison between Experimental Example 1 and Experimental Example 2, relative to the Klenow fragment, the use of the mutant of the Klenow fragment may improve a library yield and a library conversion efficiency to a certain extent. As will be seen from Experimental Examples 2 to 5, as a concentration of the SSB increases, both the library yield and the library conversion efficiency show an increasing trend.

In summary, since the SSB is added to the DNA end repair regent, the SSB may bind to the sticky ends when the sticky ends of the DNA fragments are repaired. It may be possible to make the sticky ends of the DNA fragments in the extending state without bend and knot, which may prevent a DNA super-secondary structure (e.g., a hairpin structure) from being formed. In this way, it may be conductive to binding the DNA end repair combinatorial enzyme, such as the enzyme I, to the sticky ends of the DNA fragments to promote the enzymatic reaction; and when the newly formed DNA strand bind to the sticky end at the position where the SSB binds, the SSB will fall off, which is beneficial to repair the sticky ends of the DNA fragments, improve the repair efficiency, and prevent the loss caused by a large number of DNA fragments that fail to form complete double-stranded DNA. Therefore, the library yield may be increased, and the library conversion efficiency may be improved. Moreover, in the initial stage of the whole process of the enzymatic reaction, by binding the SSB to the sticky ends of the DNA fragments, it may be possible to protect the sticky ends of the DNA fragments from undergoing an enzymatic hydrolysis reaction, so that the DNA fragments may be kept intact. Therefore, the defects, such as missing, in the DNA fragments to be sequenced during the library construction process may be avoided, and the constructed library may meet requirements for using high-throughput sequencing and back-end targeted sequencing.

The foregoing descriptions are merely specific implementations of the present disclosure. However, the protection scope of the present disclosure is not limited thereto. Changes or replacements that any person skilled in the art could conceive of within the technical scope of the present disclosure shall be included in the protection scope of the present disclosure. Therefore, the protection scope of the present disclosure shall be subject to the protection scope of the claims.

1. A deoxyribonucleic acid (DNA) end repair reagent, comprising:
a DNA end repair combinatorial enzyme; and
a single strand DNA-binding protein (SSB).

2. The DNA end repair reagent according to claim 1, wherein
the SSB is a T4 gene 32 protein.

3. The DNA end repair reagent according to claim 1, wherein
an amino acid sequence of the SSB is as shown in a sequence 1 in a sequence listing.

4. The DNA end repair reagent according to claim 1, wherein
a concentration of the SSB in the DNA end repair reagent is in a range of 0.5 μg/μL to 2 μg/μL, inclusive.

5. The DNA end repair reagent according to claim 1, wherein
the DNA end repair combinatorial enzyme includes: an enzyme I having a 5′-3′ DNA polymerase activity and a 3′-5′ DNA exonuclease activity.

6. The DNA end repair reagent according to claim 5, wherein
the enzyme I includes a Klenow fragment;
or
the enzyme I includes a mutant of the Klenow fragment.

7. The DNA end repair reagent according to claim 6, wherein
an amino acid sequence of the mutant of the Klenow fragment is as shown in a sequence 2 in a sequence listing.

8. The DNA end repair reagent according to claim 6, wherein
in a case where the enzyme I in the DNA end repair combinatorial enzyme includes the Klenow fragment, a concentration of the Klenow fragment in the DNA end repair reagent is in a range of 0.02 U/μL to 0.15 U/μL, inclusive; and
in a case where the enzyme I in the DNA end repair combinatorial enzyme includes the mutant of the Klenow fragment, a concentration of the mutant of the Klenow fragment in the DNA end repair reagent is in a range of 0.02 U/μL to 0.15 U/μL, inclusive.

9. The DNA end repair reagent according to claim 6, further comprising:
a polyethylene glycol (PEG), the PEG being selected from one or more of a PEG-4000, a PEG-6000 and a PEG-8000.

10. The DNA end repair reagent according to claim 9, wherein
a mass percentage of the PEG in the DNA end repair reagent is in a range of 8% to 25%, inclusive.

11. A deoxyribonucleic acid (DNA) end repair kit, comprising:
the DNA end repair reagent according to claim 1.

12. (canceled)

13. (canceled)

14. (canceled)

15. A deoxyribonucleic acid (DNA) library construction kit, comprising: the DNA end repair kit according to claim 11, and a DNA adapter ligation kit, wherein the DNA adapter ligation kit includes a DNA adapter ligation reagent, and the DNA adapter ligation reagent includes a Polyethylene glycol-4000 (PEG-4000).

16. A method for constructing a deoxyribonucleic acid (DNA) library, comprising:
fragmenting the genomic DNA to obtain first DNA fragments;
treating the first DNA fragments by using the DNA end repair reagent according to claim 1 to obtain second DNA fragments, the second DNA fragments being each a fragment with flush ends which is phosphorylated at 5′ ends and with adenine (A) at 3′ ends, wherein a treatment condition is: firstly treating at 15° C. to 25° C. for 10 min to 20 min, and then treating at 60° C. to 70° C. for 10 min to 20 min;
ligating sequencing adapters to the second DNA fragments to obtain adapter ligation products; and
purifying the adapter ligation products, and enriching the purified products.

17. The method for constructing the DNA library according to claim 16, wherein
ligating the sequencing adapters to the second DNA fragments, includes:
treating the second DNA fragments by using a DNA adapter ligation reagent including a Polyethylene glycol-4000 (PEG-4000), so as to ligate the sequencing adapters to the second DNA fragments.

18. The DNA end repair reagent according to claim 1, further comprising: a dNTP, wherein
the DNA end repair combinatorial enzyme further includes: an enzyme II capable of adding A to a 3′ end of DNA, and an enzyme III capable of phosphorylating a 5′ end of the DNA.

19. The DNA end repair reagent according to claim 18, wherein
the enzyme II includes a Taq DNA polymerase, and the enzyme III includes a T4 polynucleotide kinase (T4 PNK).

20. The DNA library construction kit according to claim 15, wherein
a mass percentage of the PEG-4000 in the DNA adapter ligation reagent is in a range of 8% to 25%, inclusive.
